(19) United States
(12) Patent Application Publication
(10) Pub. No.: US 2016/0146929 A1
(43) Pub. Date: May 26, 2016

(54) ULTRASOUND DIAGNOSTIC DEVICE

(71) Applicant: HITACHI ALOKA MEDICAL. LTD., Tokyo (JP)

(72) Inventors: Masanori Hisatsu, Mitaka-shi (JP); Shingo Yoshizawa, Mitaka-shi (JP)

(73) Assignee: HITACHI ALOKA MEDICAL, LTD., Mitaka-shi, Tokyo (JP)

(21) Appl. No.: 14/902,904

(22) PCT Filed: Jul. 8, 2014
(86) PCT No.: PCT/JP2014/068211

§ 371 Date: Jan. 5, 2016

(30) Foreign Application Priority Data
Jul. 10, 2013 (JP) 2013-145012

(51) Int. Cl. G01S 7/52; A61B 8/00; G10K 11/34

(52) U.S. Cl. CPC G01S 7/52047; G10K 11/346; A61B 8/4483

(57) ABSTRACT

An ultrasound diagnostic device comprises a coefficient computation unit. The coefficient computation unit computes a coefficient on the basis of phase scattering in a plurality of received signals arranged in an element array direction. Beam data to which a phasing has been added is multiplied by the coefficient. A correction unit ensures that the coefficient does not get smaller than necessary on the basis of a transmission frequency. Excessive suppression of a main lobe component is thus eliminated or reduced.

### TECHNICAL FIELD

The present invention relates to an ultrasonic diagnosis apparatus, and more particularly to processing for suppressing unwanted components such as a side lobe component contained in beam data.

### BACKGROUND ART

An ultrasonic diagnosis apparatus is an apparatus that transmits and receives ultrasound to and from an organism such as a human body and forms an ultrasonic image based on a received signal obtained by transmission and reception of the ultrasound. When transmitting and receiving ultrasound, a transmitting beam and a received beam are formed, of which the received beam will be described. Each of a plurality of received signals output from an array transducer undergo delay processing and then these delayed received signals are summated, so that beam data as a received signal which has undergone phase alignment and summation processing (delay and summation processing) can be obtained. In forming the received beam, receiving dynamic focus is generally applied for moving a receiving focus point in the depth direction in accordance with the movement of a received sample point in the depth direction.

The received signal after the phase alignment and summation contains, in addition to a signal component corresponding to a main lobe (main lobe component), various unwanted signal components which are generated by a side lobe, a grating lobe, and so on. With regard to a sequence of received signals after the delay processing and before the summation processing, the unwanted signal components contained in these signals are generally observed as a variation of phases (instantaneous amplitudes) in the element arrangement direction (channel direction). Several methods for reducing the unwanted signal components using this feature have been proposed. According to such methods, a coefficient for use in gain adjustment is computed based on a variation (or degree of uniformity) of phases in the element arrangement direction, and beam data after the phase alignment and summation is multiplied by the coefficient. Such a coefficient has a value within a range of 0 to 1, for example. The more the phases are aligned among a plurality of received signals after the delay processing, the smaller the unwanted signal component and the more the main lobe component is dominant, and therefore a greater value is computed as the coefficient. On the contrary, the more the phases vary among a plurality of received signals after the delay processing, the unwanted signal components are regarded to be relatively great, and a smaller value is computed as the coefficient.

Such a coefficient may include a CF (Coherence Factor) (see Patent Document 1, for example), a GCF (Generalized Coherence Factor) (see Non Patent Document 1, for example), an SCF (Sign Coherence Factor) (see Non Patent Document 2, for example), a GSCF (Generalized Sign Coherence Factor) (see Patent Document 2, for example), an STF (Sign Transit Factor)(see Patent Document 3, for example), a PCF (Phase Coherence Factor) (see Non Patent Document 2, for example), and the like.

### CITATION LIST

### Patent Literature

[Patent Document 1] U.S. Pat. No. 5,910,115

[Patent Document 2] JP2012-152311A

[Patent Document 3] JP2012-223430A

### Non-Patent Literature

[Non Patent Document 1] Pai-Chi Li and Meng-Lin Li. “Adaptive Imaging Using the Generalized Coherence Factor”, IEEE Transactions on Ultrasonics, Vol.50, No.2 (February 2003).

[Non Patent Document 2] Jorge Camacho, Montserrat Parrilla, and Carlos Fritsch, “Phase Coherence Imaging”, IEEE Transaction on Ultrasonics, Ferroelectrics and Frequency Control, Vol.56, No.5, (May 2009).

### SUMMARY OF INVENTION

### Technical Problems

As the above coefficient is calculated based on a change in the phase in the element arrangement direction (channel direction), even the phases of main lobe components are not aligned with each other after the phase alignment processing, if the set velocity of sound c₀ which, is a basis for calculation of delay time used in the delay processing concerning individual received signals, and the actual velocity of sound “c” in the organism differ from each other. While the sound velocity correction technique has recently become widespread, it is still difficult to completely match the velocity of sound on calculation with the actual velocity of sound.

Even in the main lobe components, misalignment in the phase is unavoidably caused to some extent in the element arrangement direction. Such a phase misalignment is greater as the transmission frequency (which is basically the same as the reception frequency) is higher. This is because the higher the transmission frequency, the faster the change in the phase on the time axis in each received signal, and therefore the greater the shift of phase in the element arrangement direction. Consequently, as the transmission frequency increases, the value of the coefficient decreases to suppress the beam data to a greater extent, leading to a problem that even the main lobe components which should not be suppressed are excessively suppressed.

### Solution to Problems

An advantage of the present invention is to prevent a main lobe component from being excessively suppressed in suppression processing of unwanted signal components in an ultrasonic diagnosis apparatus, and particularly to eliminate or alleviate effects caused by a change in the transmission frequency in the suppression processing of unwanted signal components.

An ultrasonic diagnosis apparatus according to the present invention includes a receiving unit configured to apply delay processing and summation processing to a plurality of received signals output from an array transducer composed of a plurality of transducer elements and to output beam data, a coefficient computation unit configured to compute a coefficient for adjusting a gain of the beam data while referring to all or some of the plurality of received signals after the delay processing and prior to the summation processing, and to compute the coefficient such that as a variation of phases in an element arrangement direction concerning all or some of the plurality of received signals after the delay processing and prior to the summation processing is greater, the beam data is suppressed to a greater degree, and a suppression processing unit configured to apply suppression processing to the beam data based on the coefficient. The coefficient computation unit computes the coefficient such that as a transmission frequency is higher, a degree of suppression is smaller in the suppression processing applied to the beam data.

With the above structure, based on all or some of a plurality of received signals after the delay processing and before the summation processing, a coefficient for adjusting the gain of beam data generated by the delay and summation processing (phase alignment and summation processing), that is, a coefficient for suppressing the beam data, is computed. At this time, the coefficient is computed such that the degree of suppression of the beam data is reduced more as the transmission frequency (which is generally the same as the reception frequency) is higher. More specifically, because, as the transmission frequency is higher, the instantaneous amplitudes (or phases) become misaligned even in the received signal components corresponding to a main lobe, the apparatus is configured so as to prevent the main lobe components, in addition to the unwanted signal components, from being excessively suppressed. While it is desirable to apply the above gain adjustment to the beam data after detection, the above gain adjustment may be applied to the beam data before detection.

Preferably, the coefficient computation unit computes the coefficient based on a function for obtaining the coefficient from the variation of phases, and, in accordance with the transmission frequency, an input condition of the function is changed or a parameter value in the function is changed. The computation based on the function may be implemented by a processor which operates according to a program, or may be implemented by dedicated software.

The above coefficient may include a CF (Coherence Factor), a GCF (Generalized Coherence Factor), an SCF (Sign Coherence Factor), a GSCF (Generalized Sign Coherence Factor), an STF (Sign Transit Factor), a PCF (Phase Coherence Factor), and the like. A function suitable for the coefficient which is used is adopted. It is desirable to select a method for changing the coefficient in accordance with the transmission frequency, that is, a method for correcting the characteristics of a function (characteristic correction method), in accordance with the nature of each coefficient. The characteristic correction method may include an index correction method for changing the magnitude of an index as a parameter value in the function, an offset value correction method for changing the magnitude of an offset value as a parameter value in the function, an input condition correction method for changing the number or structure of input signals to be applied to the function, and the like. If a predetermined frequency component is referred to in the spectrum concerning the amplitude distribution in the element arrangement direction for computation of a coefficient, a section correction method for changing the reference section and the like may be employed.

Preferably, the coefficient computation unit includes an input aperture adjusting unit which, in accordance with the transmission frequency, changes an input aperture for selecting a plurality of received signals to be applied to the function from among the plurality of received signals after the delay processing and prior to the summation processing, and the number of received signals to be applied to the function is changed in accordance with the transmission frequency. A shift of phases caused by a variation of the velocities of sound is smaller toward the center of an aperture. This structure limits a signal to be applied to the function to a signal in the vicinity of the center of a receiving aperture to make the variation apparently small, thereby alleviating excessive suppression with respect to the beam data. This processing can be easily implemented by selection of signals. A weight function may be applied to the input aperture.

Preferably, the input aperture is included in the receiving aperture which expands in the element arrangement direction for forming a received beam. The input aperture is separately formed from the receiving aperture. The receiving aperture is dynamically changed in accordance with a depth of a received sample point and the like. At this time, the receiving aperture may be changed with the input aperture. In any case, the input aperture has a size which is the same as or smaller than that of the receiving aperture. However, during computing, the input aperture may be virtually larger than the receiving aperture. Preferably, the input aperture is changed in accordance with the depth of the received sample point on the received beam. When variable aperture control is performed in synchronism with reception dynamic focus, the input aperture is dynamically varied accordingly. It is also possible to make the receiving aperture correspond to the input aperture, and in this case, the size of the receiving aperture at each depth is changed in accordance with the transmission frequency.

Preferably, the coefficient computation unit includes a parameter value changing unit which changes, in accordance with the transmission frequency, an index or an offset value within the function as the parameter value. Correction of an index and an offset value can change the characteristics of a function easily.

Preferably, the function is a function for computing the coefficient based on a direct current vicinity component contained in an amplitude distribution in the element arrangement direction that is formed based on all or some of the received signals after the delay processing and prior to the summation processing, and the coefficient computation unit includes a section changing unit that changes a size of a section defining the direct current vicinity component as the parameter value based on the transmission frequency. With this correction, the sensitivity of the unwanted signal component is changed to thereby reduce or prevent excessive suppression of the main lobe component.

### BRIEF DESCRIPTION OF DRAWINGS

FIG. 1 is a block diagram illustrating a principal structure of an ultrasonic diagnosis apparatus according to the present invention.

FIG. 2 is a diagram illustrating a first example of a coefficient computation unit.

FIG. 3 is a diagram for explaining effects of index.

FIG. 4 is a diagram illustrating a second example of the coefficient computation unit.

FIG. 5 is a diagram for explaining a variation of a reference aperture.

FIG. 6 is a diagram illustrating a third example of the coefficient computation unit.

FIG. 7 is a diagram for explaining effects of an offset value.

FIG. 8 is a diagram illustrating a fourth example of the coefficient computation unit.

FIG. 9 is a diagram illustrating the vicinity of DC in a spectrum.

FIG. 10 is a diagram illustrating a fifth example of the coefficient computation unit.

FIG. 11 is a diagram illustrating a sixth example of the coefficient computation unit.

FIG. 12 is a diagram illustrating a seventh example of the coefficient computation unit.

FIG. 13 is as diagram illustrating an eighth example of the coefficient computation unit.

FIG. 14 is a diagram illustrating a ninth example of the coefficient computation unit.

FIG. 15 is a diagram illustrating a tenth example of the coefficient computation unit.

FIG. 16 is a diagram illustrating an eleventh example of the coefficient computation unit.

FIG. 17 is a diagram illustrating a twelfth example of the coefficient computation unit.

FIG. 18 is a diagram illustrating a thirteenth example of the coefficient computation unit.

FIG. 19 is a diagram illustrating a relationship between a receiving aperture and an input aperture.

FIG. 20 is a diagram for explaining a variation of the input aperture in accordance with the depth.

### DESCRIPTION OF EMBODIMENTS

A preferred embodiment of the present invention will be described with reference to the drawings.

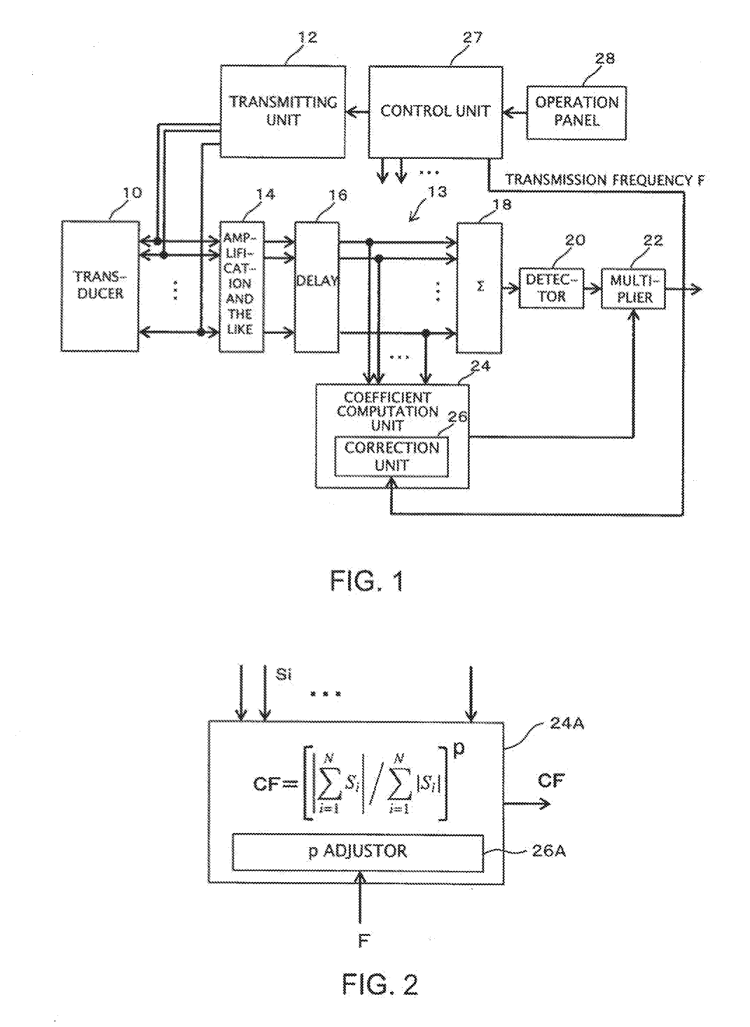

FIG. 1 is a block diagram illustrating an ultrasonic diagnosis apparatus according to a preferred embodiment of the present invention. This ultrasonic diagnosis apparatus is an apparatus which is used in a medical field and forms an ultrasonic image based on a received signal obtained by transmitting and receiving ultrasound to and from an organism. In the present embodiment, the ultrasonic diagnosis apparatus has a function of suppressing an unwanted signal component.

Referring to FIG. 1, reference numeral 10 denotes an array transducer. The array transducer 10 is formed of a plurality of transducer elements. Each transducer element convers an electrical signal to ultrasound, or converts ultrasound to an electrical signal. While in the present embodiment the array transducer 10 is a 1D array transducer, a 2D array transducer may be used. The array transducer 10 forms an ultrasound beam, which is electronically scanned. Electronic linear scanning, electronic sector scanning, and the like are known as electronic scanning methods.

A transmitting unit 12 is a transmitting beam former. At the time of transmission, the transmitting unit 12 applies a plurality of transmitting signals having a predetermined delay relationship to the array transducer 10, such that a transmitting beam is formed on the array transducer 10. The transmitting unit 12 is a transmitting processor or a transmitting circuit. At the time of reception, receiving a reflected wave from within the organism by the array transducer 10, the array transducer 10 outputs a plurality of received signals to a receiving unit 13.

The receiving unit 13 is a received beam former, and executes delay processing with respect to a plurality of received signals and then applies summation processing to the delayed received signals, thereby generating beam data corresponding to a received beam. The receiving unit 13 is a receiving processor or a receiving circuit. According to the present embodiment, the receiving unit 13 includes a pre-processing circuit 14, a delay circuit 16, a summation circuit 18, and the like, as will be described below.

The pre-processing circuit 14 is composed of a plurality of processing devices provided corresponding to a plurality of received signals, and each processing device is composed of a preamplifier, an A/D converter, a gain adjuster, and the like. Weighting processing within a receiving aperture is executed in this pre-processing circuit 14.

The delay circuit 16 is composed of a plurality of delay devices provided corresponding to a plurality of received signals. Each delay device executes processing for delaying a received signal by an amount of delay time which is set by a transmitting/receiving control unit. The delay time is calculated in advance in accordance with a location of a received focus point (received sample point), a beam steering direction, and the like.

The summation processing circuit 18 executes summation processing with respect to a plurality of received signals having undergone the delay processing, thereby obtaining beam data as a received signal after the phase alignment and summation. The summation processing circuit 18 is composed of one or a plurality of adders, for example. The received signal output from the receiving unit 13, that is, beam data, undergoes detection processing in a detection unit 20, and the beam data after the detection processing is transmitted, via a multiplier 22, to an image processing circuit (not shown) on the downstream side. The detection unit 20 is a detection circuit.

The multiplier 22 functions as a gain adjusting circuit or an unwanted signal component suppression circuit. The multiplier 22 is a multiplication circuit. A coefficient which is computed by a coefficient computation unit 24 which will be described below is multiplied by the beam data in the multiplier 22, thereby suppressing the unwanted signal component. Here, the coefficient corresponds to a gain value. However, a coefficient which represents a degree of attenuation of a signal may alternatively be computed. As described above, a difference between the velocity of sound on which delayed data calculation is based and the actual velocity of sound within an organism causes a shift in the phases between the received signals during the phase alignment and summation processing, and this shift increases as the transmission frequency becomes higher. If the signal suppression processing based on the coefficient as described above is executed in such a case, there may arise a problem that even a main lobe component, that is, a true signal component, is excessively suppressed, particularly when the transmission frequency is increased. To address such a problem, according to the present embodiment, the coefficient computation unit 24 includes a correction unit 26.

As illustrated in FIG. 1, a plurality of received signals are extracted separately (from diverged paths) between the delay circuit 16 and the summation circuit 18, and the plurality of extracted received signals are input to the coefficient computation unit 24. The coefficient computation unit 24 is implemented by dedicated hardware or a processor which operates according to a program. The coefficient computation unit 24, based on the plurality of received signals, computes the above-described coefficient in accordance with (based on) a variation of the phase in the element arrangement direction (that is, a distribution of amplitude). According to the present embodiment, the correction unit 26 is provided to prevent excessive signal suppression in accordance with the transmission frequency, and this correction unit 26 variably sets characteristics of a function for computing the coefficient. There are a plurality of functions for computing the coefficient and a plurality of methods for correcting the degree of reduction, which will be described below. Here, the coefficient computation unit 24, for each received sample point at each depth, refers to the amplitude waveform in the element arrangement direction, based on which the coefficient is computed. The element arrangement direction refers to a direction in which the received signals are arranged. Observation of a variation of the phase, i.e., an instantaneous amplitude, in such a direction, enables an ex post facto assessment as to whether or not the delay processing result is appropriate. The correction unit 26 is a correction processor or a correcting circuit.

Referring to FIG. 1, the control unit 27 is composed of a CPU which executes an operation program. In other words, the control unit 27 is a control processor. The control unit 27 controls the operation of each of the constituent elements illustrated in FIG. 1, and particularly controls the transmitting and receiving processing. An operation panel 28 is formed of a keyboard, track ball, and the like, and a parameter value or the like input by a user can be input to the control unit 27 using the operation panel 28. According to the present embodiment, information representing the transmission frequency selected automatically or by a user is transmitted from the control unit 27 to the correction unit 26. The correction unit 26 may be implemented as a function of the control unit 27.

The coefficients (gain coefficients) for suppressing unwanted signal components include, as described above, CF, GCF, SCF, GSCF, STF, PCF, and the like, each of which is a coefficient corresponding to a magnitude of a variation of the amplitude waveform (amplitude distribution, amplitude profile) in the element arrangement direction. Methods for changing the characteristic (degree of suppression) of a function for computing these coefficients in accordance with the transmission frequency include an index correction method, an input aperture correction method, an offset value correction method, a reference band correction method, and the like. It is desirable that a correction method which matches properties and conditions of the coefficient is selectively adopted.

The index correction method is a method for changing a value of the index in a function to adjust the degree of suppression in accordance with the transmission frequency. The input aperture correction method is a method for changing the arrangement (particularly the number of signals) of a received signal sequence to be applied to a function in accordance with the transmission frequency to decrease the apparent variation, thereby adjusting the degree of suppression. The offset value correction method is a method for summing an offset value in the function and changing the magnitude of offset value in accordance with the transmission frequency, thereby adjusting the degree of suppression. The reference band correction method is a method for varying the size of a section (band) to be referred to on the spectrum of the amplitude waveform in the element arrangement direction in accordance with the transmission frequency, thereby adjusting the degree of suppression. Any methods other than the above methods may also be adopted.

Each of the coefficients and a representative correction method (index correction method) will be described below.

The CF is calculated according to the following Expression (1), for example. In the expression, “Si” denotes the i-th received signal after the delay processing and prior to the summation processing. The “i” is an integer from 1 to N. N received signals correspond to a receiving aperture, for example. The CF, similar to other coefficients, is sequentially computed for each received sample point at each depth.

### Mathematical Expression 1

In the above Expression (1), the denominator is a sum of absolute values of N received signals, in which a sign of each received signal is not taken into consideration. The denominator is provided for the purpose of normalization. On the other hand, the numerator in the above Expression (1) is an absolute value of a sum of the N received signal, in which signs are taken into consideration for summation. Accordingly, the numerator represents a variation (non-uniformity) of the phases of the N received signals.

The index correction method described above can be used to change the characteristic of a function for computing this CF in accordance with the transmission frequency, for example. In this case, an index “p” in the function shown in the following Expression (2) is utilized.

### Mathematical Expression 2

The GCF is calculated according to the following Expression (3), for example. The denominator in Expression (3) represents a total power value concerning the spectrum of the amplitude waveform in the element arrangement direction, and the numerator in this expression represents a power value of a DC vicinity component including a DC component in the same spectrum.

### Mathematical Expression 3

If the above amplitude waveform is completely flat, the power will concentrate on DC in the spectrum, whereas if there is a variation in the amplitude waveform, the spectrum will expand toward the high frequency side. It is therefore possible to assess the degree of variation of the amplitude waveform by the power value of the DC vicinity component. The DC vicinity is defined as a range from DC to a predetermined frequency, whose width (band) is designated by M which will be described below, for example. If the index correction method is applied to the above Expression (3), the following Expression (4) is utilized.

### Mathematical Expression 4

It is possible to correct the degree of suppression of the beam data by changing “p” in Expression (4) in accordance with the transmission frequency. If the reference band correction method is adopted, the magnitude of the above M is changed by the transmission frequency.

The SCF is calculated according to the following Expression (5), for example. Here, a function in which the index correction method has been incorporated is shown. In Expression (5), “i” denotes the number of the received signal, which, in the following example, ranges from 0 to N−1.

### Mathematical Expression 5

In the above Expression (5), “bi” is defined by the following Expression (6). More specifically, “bi” is a binarization result of the received signal.

### Mathematical Expression 6

The above Expression (5) includes calculation of an integration value (mean value) as a variation concerning a signal sequence after binarization.

The GSCF is defined according to the following Expression (7), for example.

### Mathematical Expression 7

In GSCF, each received signal is binarized. By computing, under this precondition, [power value of DC vicinity component]/[total power value of spectrum], similar to the above GCF, GSCF is obtained. “N” denotes the number of received signals, and “M” denotes a parameter value which defines the DC vicinity as described above. If the index correction method is applied to this GSCF, a function represented by the following Expression (8) is utilized.

### Mathematical Expression 8

The STF is defined according to the following Expression (9). In, the following Expression (9), the depth “k” of a received sample point is explicitly indicated. Also, an index “q” in accordance with the index correction method is incorporated. In the present embodiment, this index “q” is changed in accordance with the transmission frequency.

### Mathematical Expression 9

A(k) in the above Expression (9) is defined according to the following Expression (10).

### Mathematical Expression 10

Here, ci(k) in the above Expression (10) is defined as in the following Expression (11).

### Mathematical Expression 11

The above Expression (11) is a sensor for inversion of a sign: if a location of sign inversion is detected in the element arrangement direction, ci(k) is set to 1. Concerning the depth “k”, the number of sign determinations in the element arrangement direction represents the degree of variation of the amplitude waveform in the same direction, and STF which reflects such a degree is defined as in the above Expression (9). In the above example description, the representative coefficients have been described, and description of the PCF and other coefficients will be omitted.

With reference to FIGS. 2 to 18, specific example structures of the coefficient computation unit described above will be described.

FIG. 2 illustrates a first example coefficient computation unit. The coefficient computation unit 24A illustrated in FIG. 2 executes the above Expression (2). The coefficient computation unit 24A includes a “p” adjustor 26A which adjusts the index “p”. The “p” adjustor 26A variably sets the index “p” based on the transmission frequency F. The “p” adjustor 26A functions as a parameter changing unit, which is composed of a processor or a circuit. Other adjustors which will be described below also function as parameter changing units which are composed of a processor or a circuit.

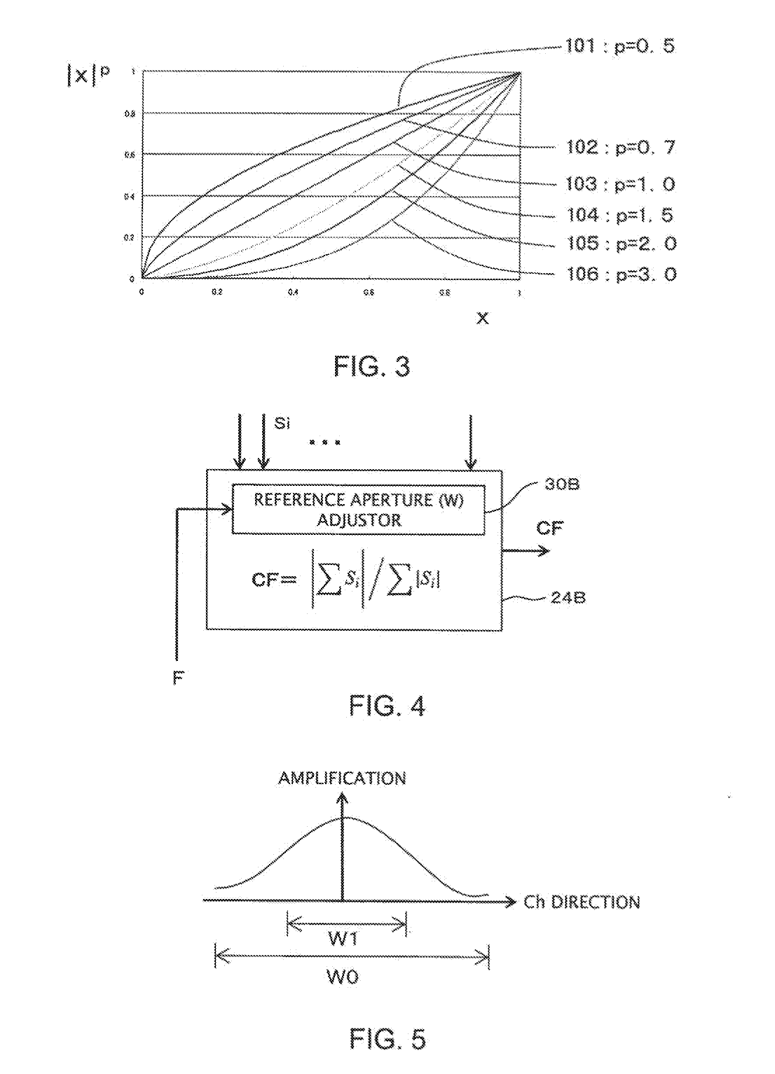

FIG. 3 illustrates the relationship between x and |x|^{p} in a graph form. A graph 101 indicates a case in which p is 0.5; a graph 102 indicates a case in which p is 0.7; a graph 103 indicates a case in which p is 1.0; a graph 104 indicates a case in which p is 1.5; a graph 105 indicates a case in which p is 2.0; and a graph 106 indicates a case in which p is 3.0. As illustrated, variable setting of the value of “p” enables correction of the characteristic of the function in the above Expression (2), that is, enables manipulation of the value of the coefficient CF in accordance with the transmission frequency. This structure can make the value of the coefficient less reduced as the transmission frequency is higher, thereby avoiding a problem that the main lobe component is reduced more than necessary. Conversely, it is possible to configure the apparatus such that the index is set to a greater value when the transmission frequency is low to thereby suppress the unwanted signal component more positively.

FIG. 4 illustrates a second example coefficient computation unit. The coefficient computation unit 24B executes Expression (1) described above. The coefficient computation unit 24B includes a reference aperture adjustor 30B which variably sets the number of received signals, i.e., the size of the input aperture in accordance with the transmission frequency F and which is one embodiment of the correction unit illustrated in FIG. 1.

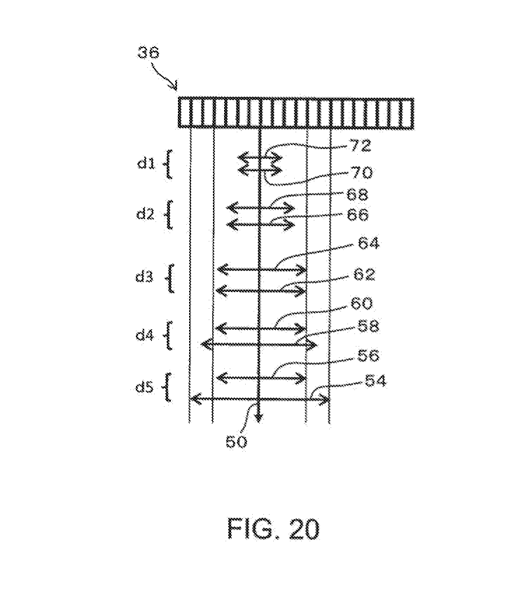

As illustrated in FIG. 5, for example, the reference aperture adjustor described above sets a greater input aperture W0 when the transmission frequency is low, and sets a smaller input aperture W1 when the transmission frequency F is increased. FIG. 5 illustrates, in the upper level thereof, an amplitude distribution in the element arrangement direction, in which the center of the amplitude distribution corresponds to the center of the main beam. The input aperture, i.e., the reference aperture, may be changed continuously in accordance with the magnitude of the transmission frequency or may be changed stepwise. The input aperture is generally set within the receiving aperture, and does not effectively exceed the receiving aperture. This will be described below with reference to FIGS. 19 and 20. With the adjusting method of the input aperture described above, manipulation of the number of input signals can vary the degree of apparent variation to thereby correct the effects of a function. This can advantageously ease the problem that the main lobe component is unnecessarily reduced when the transmission frequency is high.

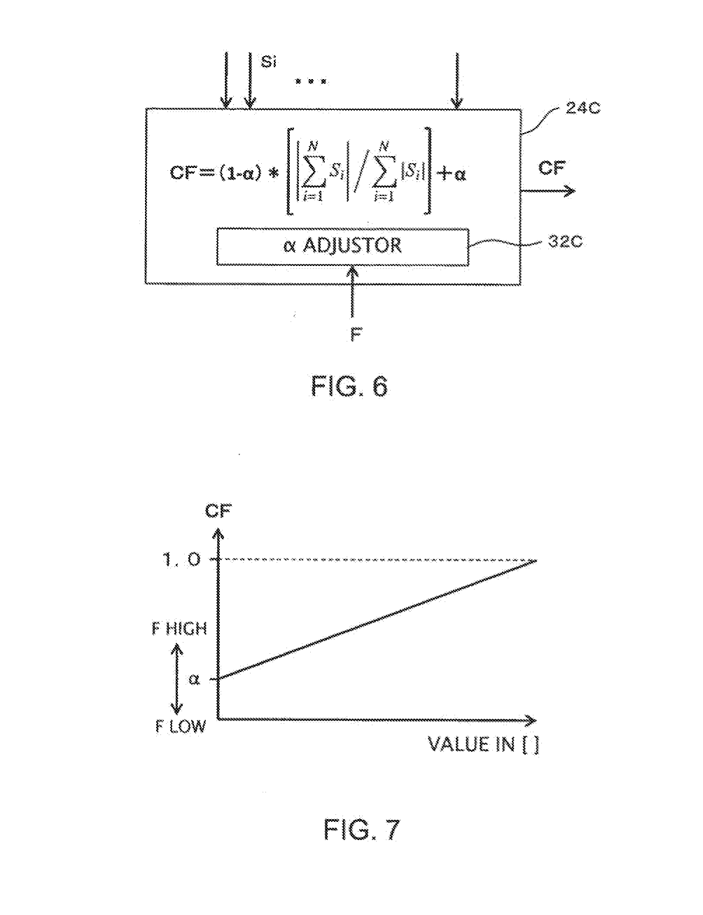

FIG. 6 illustrates a third example coefficient computation unit. The coefficient computation unit 24C is a module for computing a sum of the result obtained by Expression (1), which is a base, added by an offset value. In FIG. 6, the offset value is “α”, and the part corresponding to the right-hand side of Expression (1) is multiplied by a weight (1−α). The coefficient computation unit 24C includes an “α” adjustor 32C which variably sets the offset value “α” as a parameter value based on the transmission frequency F. More specifically, the “α” adjustor 32C sets the offset value “α” such that the offset value “α” is greater as the frequency F of the transmitting signal is higher, in order to implement a reduction degree correction means.

This is illustrated in FIG. 7, in which the horizontal axis indicates a value in the bracket in the computational expression shown in FIG. 6, and the vertical axis indicates the coefficient CF. By varying the offset value “α” in accordance with the transmission frequency F, it is possible to manipulate the inclination and contact point of the linear characteristic illustrated in FIG. 7. This can lead to reduction or prevention of excessive suppression of the main lobe component when the transmission frequency is high. The apparatus may be configured such that the value of “α” can be variably set by a user or the value of “α” can be automatically determined based on the image quality, signal quality, and the like.

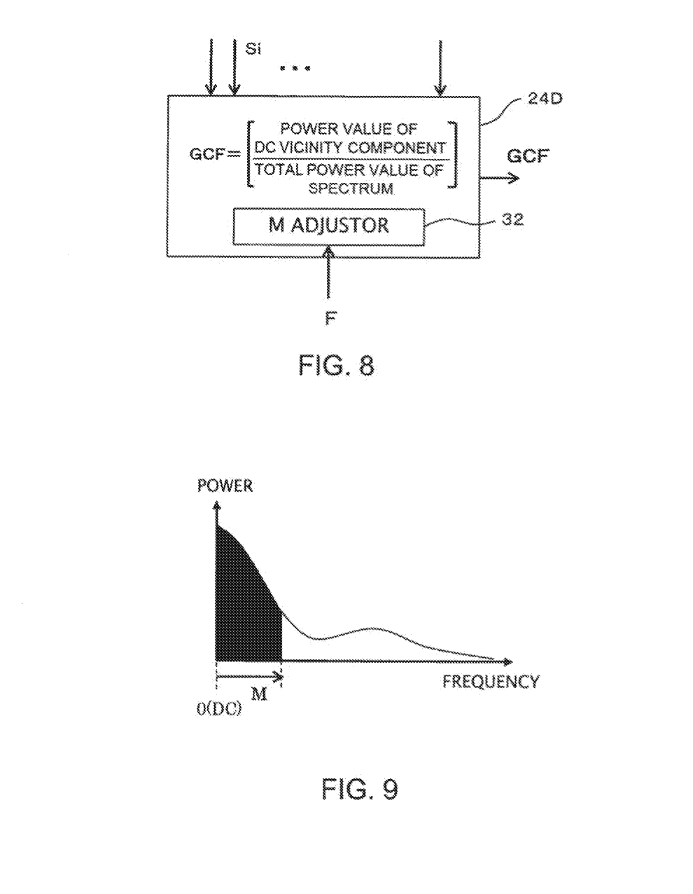

FIG. 8 illustrates a fourth example coefficient computation unit. The coefficient computation unit 24D executes Expression (3) described above. The coefficient computation unit 24D includes an M adjustor 32, which variably sets the band M for defining the DC vicinity in accordance with the transmission frequency F and functions as a section changing unit.

Specifically, FIG. 9 illustrates a spectrum of the amplitude waveform in the element arrangement direction, in which the horizontal axis indicates a frequency and the vertical axis indicates a power for each frequency. The left end of the frequency axis corresponds to DC. If the signal waveform in the element arrangement direction is flat, such as in a completely straight line, all energies would concentrate on DC in the spectrum, whereas if there is a variation or change in the signal waveform, the spectrum will expand toward the higher side on the frequency axis. As, in such a case, the DC vicinity component (the solid portion in FIG. 9) varies in accordance with the degree of variation, GCF is set as the coefficient by referring to the DC vicinity component(?). In this case, the M adjustor variably sets the band M for defining the DC vicinity in accordance with the transmission frequency. More specifically, the M adjustor increases M as the transmission frequency is higher. As this structure enables manipulation of a ratio of the area of the DC vicinity in relation to the area of the whole spectrum, it is possible to ease or eliminate the problem that the main lobe component is reduced more than necessary when the transmission frequency is high.

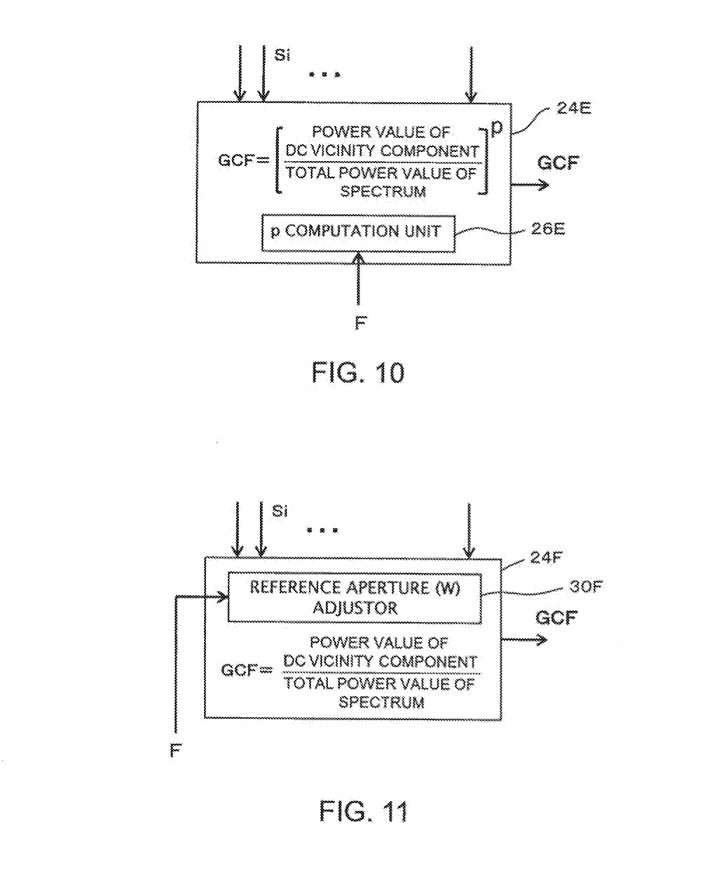

FIG. 10 illustrates a fifth example coefficient computation unit. The coefficient computation unit 24E executes Expression (4) described above. The coefficient computation unit 24E includes a “p” computation unit 26E which variably sets an index “p” in accordance with the transmission frequency. This structure can implement the index correction method represented by Expression (4).

FIG. 11 illustrates a sixth example coefficient computation unit. The coefficient computation unit 24F executes Expression (3) described above, to which the input aperture correction method is applied. Specifically, the coefficient computation unit 24F includes a reference aperture adjustor 30F which functions as an input aperture adjustor unit, and the reference aperture adjustor 30F variably sets the input aperture, i.e., reference aperture, based on the transmission frequency F. As this structure enables manipulation of the number of input signals in the function indicated in Expression (3) described above, that is, enables reduction in the apparent degree of variation, for example, it is possible to ease the problem of excessive suppression of the main lobe component when the transmission frequency is high.

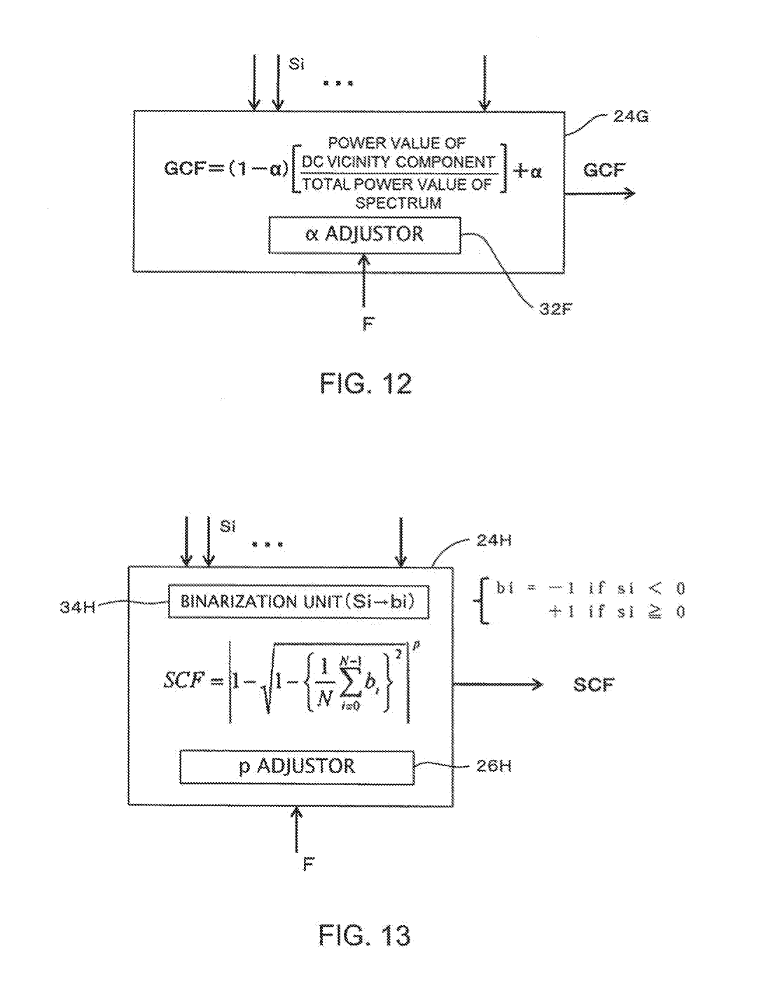

FIG. 12 illustrates a seventh example coefficient computation unit. The coefficient computation unit 24G computes a function when the offset value variable method is applied to Expression (3) described above, in which case, the offset value “α” is variably set by an “α” adjustor 32F. The “α” adjustor 32F variably sets the offset value “α” in accordance with the transmission frequency F.

FIG. 13 illustrates an eighth example coefficient computation unit. The coefficient computation unit 24H executes Expression (5) described above, that is, computes SCF as the coefficient. As illustrated, the coefficient computation unit 24H includes a binarization unit 34H and a “p” adjustor 26H. The binarization unit 34H executes Expression (6) described above. The “p” adjustor 26H variably sets an index “p” based on the transmission frequency F as one embodiment of the reduction degree correction means. The binarization unit 34H, as well as the binarization units which will be descried below, is a processor or a circuit.

With the above structure, it is possible to suppress the degree of reduction of SCF to thereby address the excessive reduction in the main lobe component when the transmission frequency F is high.

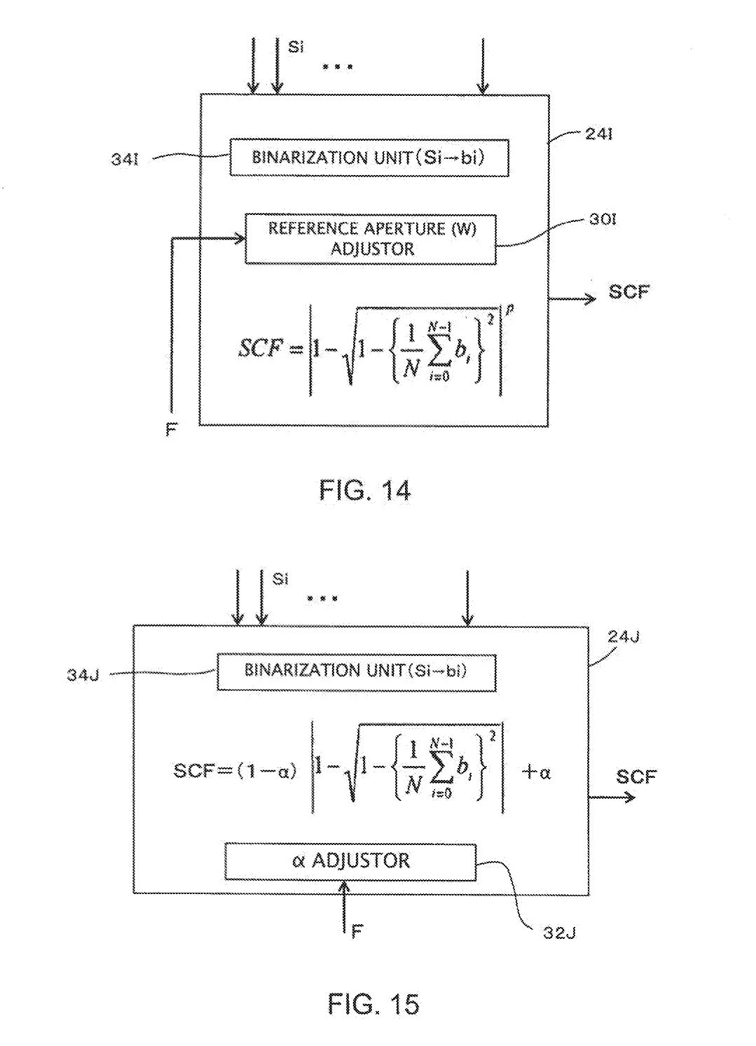

FIG. 14 illustrates a ninth example coefficient computation unit. The coefficient computation unit 24I calculates the SCF described above and includes a binarization unit 34I and a reference aperture adjustor 30I. The reference aperture adjustor 30I constitutes one embodiment of the correction unit, which manipulates the number of input signals to be applied to the function for computing the SCF to thereby adjust an apparent variation, thereby changing the characteristics of the function of the SCF.

FIG. 15 illustrates a tenth example coefficient computation unit. While the coefficient computation unit 24J, similar to the above example, calculates the SCF, modification based on the offset correction method described above is applied in the function for computing the SCF. The coefficient computation unit 24J, similar to the above example, includes a binarization unit 34J and an “α” adjustor 32J, and the “α” adjustor 32J variably sets the offset value “α” based on the transmission frequency F.

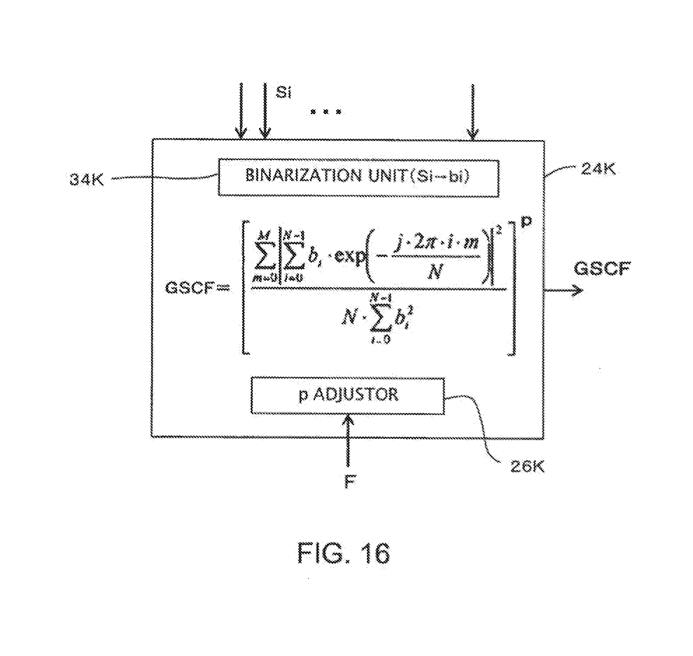

FIG. 16 illustrates an eleventh example coefficient computation unit. The coefficient computation unit 24K computes GSCF based on Expression (7) described above. As illustrated, the coefficient computation unit 24K includes a binarization unit 34K and a “p” adjustor 26K. As described above, GSCF is modification of GCF, that is, a signal obtained by converting an input signal to a binary signal. The “p” adjustor 26K variably sets the index “p” in accordance with the transmission frequency F. With this structure, it is possible to ease a problem including excessive suppression of the main lobe component.

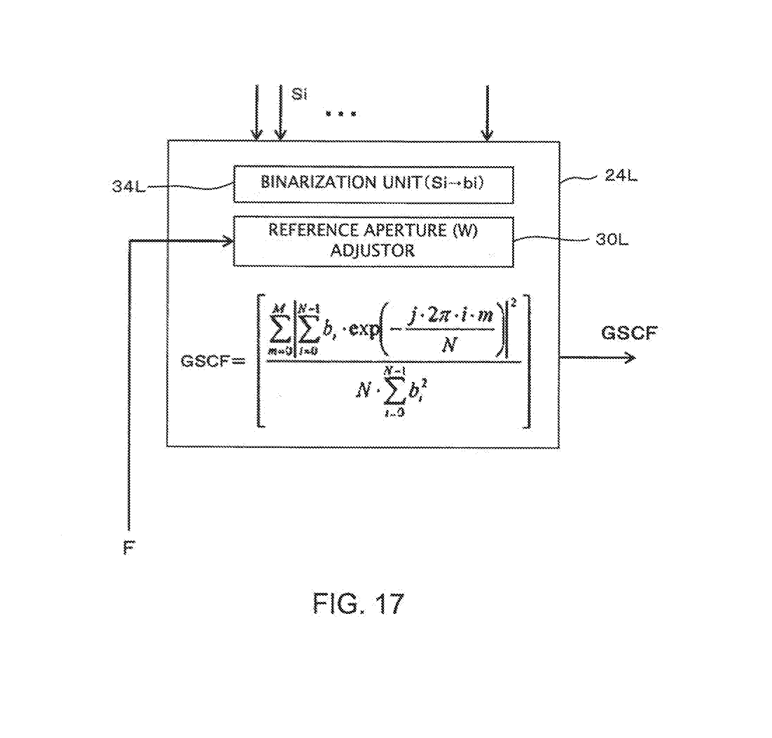

FIG. 17 illustrates a twelfth example coefficient computation unit. The coefficient computation unit 24L, similar to above example, computes GSCF, and includes, for this purpose, a binarization unit 34L. A reference aperture adjustor 30L is provided as an adjusting unit for variably setting the input aperture as a reference aperture based on the transmission frequency F.

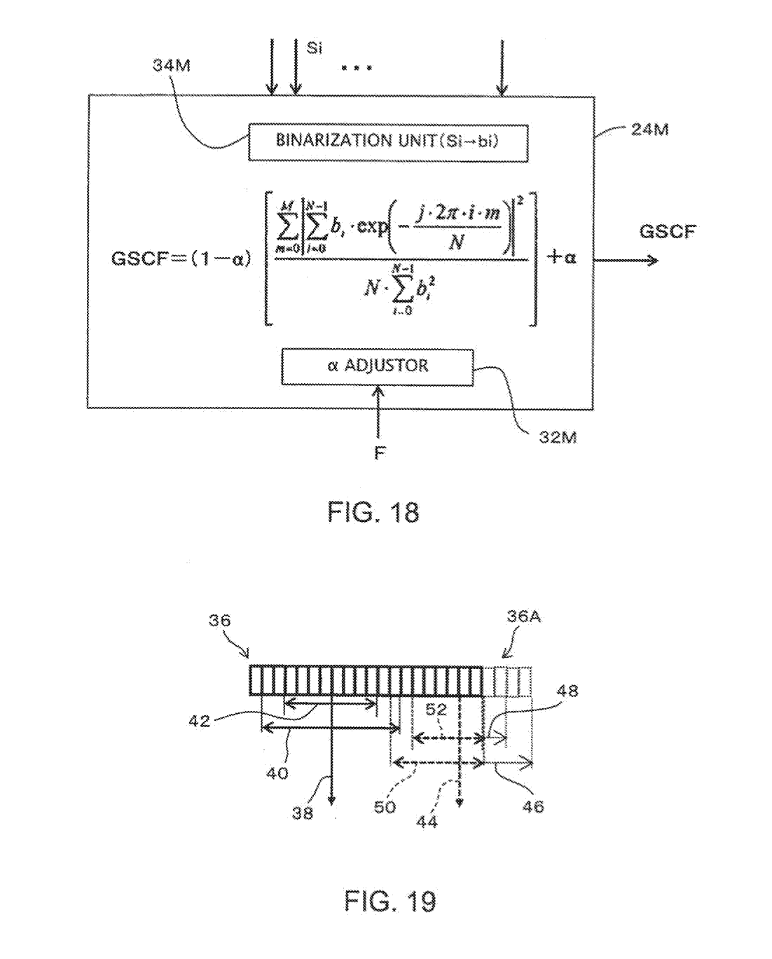

FIG. 18 illustrates a thirteenth example coefficient computation unit. The coefficient computation unit 24M, similar to the above examples, computes GSCF. Specifically, in this example, an offset “α” is incorporated with respect to the function for computing GSCF. The function computation unit 24M includes a binarization unit 34M for calculating GSCF and an “α” adjustor 32M constituting the reduction degree adjusting means. The “α” adjustor 32M variably sets the offset “α” based on the transmission frequency F.

With reference to FIGS. 19 and 20, a relationship between the receiving apertures and the input apertures (reference apertures) will be described.

Referring to FIG. 19, an array transducer 36 is composed of a plurality of transducer elements arranged along a straight line. An ultrasound beam 38, in this example, represents a transmitting beam and a received beam, and is electronically linear scanned. With this ultrasound beam 38 being a center axis, a receiving aperture 40 is set. Specifically, received signals from a plurality of receiving elements forming the receiving aperture 40 are to undergo the phase alignment and summation processing. On the other hand, an input aperture is designated by reference numeral 42. The input aperture 42 is a fixed aperture with the ultrasound beam 38 being used as the center, and the size of the input aperture 42 is variably set in accordance with the transmission frequency as described above.

The input aperture 42 is equivalent to or is set within the receiving aperture 40. Specifically, the input aperture 42 adjusts the number of reference signals in the sequence of received signals actually obtained. A state in which the ultrasound beam is electronically scanned and the ultrasound beam has reached an end portion is illustrated as denoted by numeral 44. A receiving aperture 46 is similarly set and an input aperture 48 is also set. In this case, control is performed under the assumption that virtual transducer 36A is apparently present with respect to the end portion of the array transducer 36. However, an actually effective receiving aperture is within the range indicated by reference numeral 50, and an effective input aperture is within the range indicated by reference numeral 52. In this case, one end of each of the receiving aperture and the input aperture is aligned with one end of the array transducer 36. Of course, a control example illustrated in FIG. 19 is only one example. In any case, according to the present embodiment, the receiving aperture and the input aperture are set independently from each other, and are also controlled independently of each other in accordance with the objects thereof.

FIG. 20 illustrates a change of the receiving aperture and the input aperture in accordance with the depth. An ultrasound beam 50 is shown in the direction orthogonal to the array transducer 36. The direction indicated by the ultrasound beam 50 corresponds to the depth direction. FIG. 20 shows five depths d1 to d5. For the sake of convenience, starting from the deepest level, at a depth d5, a full aperture 54 is set as the receiving aperture, within which an input aperture 56 is set. At a depth d4, a slightly smaller receiving aperture 58 is set, and within the range of the receiving aperture 58, an input aperture 60 is set. At these deep portions d4 and d5, however, the sizes of the input apertures 56 and 60 are maintained. At an intermediate depth d3 which is slightly shallower, in this example, a receiving aperture 62 corresponds to an input aperture 64. The input aperture 64, however, also corresponds to the input apertures 56 and 60 described above. While at a further shallow depth d2, a receiving aperture 66 and an input aperture 68 similarly correspond to each other, they are set within a smaller range than that of the receiving aperture and the input aperture that are set at the deeper portions. This is also the case at the shallowest depth dl, where a receiving aperture 70 and an input aperture 72 correspond to each other, but they are set within a smaller range than those of the receiving apertures and the input apertures that are set at deeper portions.

As described above, according to the present embodiment, the receiving aperture and the input aperture are set independently, or the size of each of the receiving aperture and the input aperture is set in accordance with the object thereof and depending on the depth. In the control example illustrated in FIG. 20, the size of the input range is variably set in accordance with the magnitude of the transmission frequency, as described above. When the transmission frequency is high, for example, the size of the input aperture is decreased at each depth, to thereby apparently decrease the variation to be referred to, so that it is possible to prevent the coefficient value from being excessively decreased.

As, in all of the various structural examples described above, the magnitude of the coefficient can be manipulated in accordance with the transmission frequency, it is possible to eliminate or ease the problem of excessively suppressing the main lobe component together with suppression of the unwanted signal component. Consequently, the quality of an ultrasonic image can be maintained or increased.

While the structural example illustrated in FIG. 1 does not include a sound velocity correction unit, such a circuit may be additionally provided to implement control based on the velocity of sound within an organism when computing transmitting and receiving delay data. In such a case, as the velocities of sound differ slightly in various portions within the organism, it is similarly desirable to apply the correction in accordance with the transmission frequency as described above.

In the structure illustrated in each drawing, in place of a plurality of processors, a single processor which executes a plurality of functions of the plurality of processors may be provided. Alternatively, in place of a plurality of circuits, a single circuit which executes a plurality of functions provided by the plurality of circuits may be provided. Conversely, in place of an individual processor, a plurality of processors which execute the function of the individual processor may be provided, or, in place of an individual circuit, a plurality of circuits which execute the function of the individual circuit may be provided.

1. An ultrasonic diagnosis apparatus, comprising:
a receiving unit configured to apply delay processing and summation processing to a plurality of received signals output from an array transducer composed of a plurality of transducer elements and to output beam data;
a coefficient computation unit configured to compute a coefficient for adjusting a gain of the beam data while referring to all or some of the plurality of received signals after the delay processing and prior to the summation processing, the computation unit computing the coefficient such that as a variation of phases in an element arrangement direction concerning all or some of the plurality of received signals after the delay processing and prior to the summation processing is greater, the beam data is suppressed to a greater degree; and
a suppression processing unit configured to apply suppression processing to the beam data based on the coefficient,
the coefficient computation unit computing the coefficient such that as a transmission frequency is higher, a degree of suppression is smaller in the suppression processing applied to the beam data.

2. The ultrasonic diagnosis apparatus according to claim 1, wherein
the coefficient computation unit computes the coefficient based on a function for obtaining the coefficient from the variation of phases, and
in accordance with the transmission frequency, an input condition of the function is changed or a parameter value in the function is changed.

3. The ultrasonic diagnosis apparatus according to claim 2, wherein
the coefficient computation unit comprises an input aperture adjusting unit, the input aperture adjusting unit, in accordance with the transmission frequency, changing an input aperture for selecting a plurality of received signals to be applied to the function from among the plurality of received signals after the delay processing and prior to the summation processing, and
the number of received signals to be applied to the function is changed in accordance with the transmission frequency.

4. The ultrasonic diagnosis apparatus according to claim 3, wherein
the input aperture is an aperture included in a receiving aperture expanding in the element arrangement direction for forming a received beam.

5. The ultrasonic diagnosis apparatus according to claim 4, wherein
the input aperture is changed in accordance with a depth of a received sample point on the received beam.

6. The ultrasonic diagnosis apparatus according to claim 2, wherein
the coefficient computation unit includes a parameter value changing unit, the parameter value changing unit changing, in accordance with the transmission frequency, an index or an offset value within the function as the parameter value.

7. The ultrasonic diagnosis apparatus according to claim 2, wherein
the function is a function for computing the coefficient based on a direct current vicinity component contained in an amplitude distribution in the element arrangement direction that is formed based on all or some of the received signals after the delay processing and prior to the summation processing, and
the coefficient computation unit comprises a section changing unit that changes a size of a section defining the direct current vicinity component as the parameter value based on the transmission frequency.
